(19) United States
(12) Patent Application Publication
(10) Pub. No.: US 2018/0071932 A1
(43) Pub. Date: Mar. 15, 2018

(54) NOVEL RAZOR COATING

(71) Applicants: Triantafyllos Tafas, Rocky Hill, CT (US); Despoina Tafa, Athens (GR); Ioannis Tzovenis, Athens (GR)

(72) Inventors: Triantafyllos Tafas, Rocky Hill, CT (US); Despoina Tafa, Athens (GR); Ioannis Tzovenis, Athens (GR)

(21) Appl. No.: 15/699,759

(22) Filed: Sep. 8, 2017

Related U.S. Application Data
(60) Provisional application No. 62/385,431, filed on Sep. 9, 2016.

(51) Int. Cl. B26B 21/44; A61K 8/9711; A61Q 9/02; A61K 8/9722; A61K 8/9717

(52) U.S. Cl. CPC B26B 21/443; B26B 21/446; A61K 8/9711; B26B 21/06; A61K 8/9722; A61K 8/9717; A61Q 9/02

(57) ABSTRACT

The invention comprises a modified razor combining at least one cutting blade and at least one lubricating strip for engage the skin to apply a selection of at least one algae extract for protecting and reinforcing the skin. Further, the razor may comprise a soap/foaming strip for additional cleansing and hydrating properties. Another optional inventive aspect is the hollow razor handle which may filled with balm combined with an algae composition. The application of the balm from the handle may be achieved by a dispensing wheel so as to extrude and apply the balm to the skin.

### RELATED APPLICATION

The application claims the benefit of priority of U.S. Provisional Application 62/385,431 filed Sep. 9, 2016, which is incorporated herein by reference in its entirety.

### BACKGROUND OF THE INVENTION

### Field of the Invention

This invention generally relates to a novel shaving head comprising a lubricating or skin care strip for razor blades; more specifically, the razor head associated strip relates to lubricating coatings composed of discrete packets of extracts obtained from suitable compositions or mixtures of algae comprising antioxidant and anti-inflammatory properties.

### Description of the Related Art

Many conventional razor heads combine at least two slender razor blades mounted in a razor blade housing attached to a handle through a secure joint portion optionally by an immobile rigidly or mobile ball bearing rotatably connected. It was discovered that the arrangement provided a shaving action exerting considerable dry rubbing friction and consequently irritating the shaved skin. It is an object of this invention to overcome the problems of both skin irritation and uneven shaving. A common razor or shaving instrument is found in the form of a disposable shaver typically comprising a moulded shaver head containing one or more parallel closely spaced razor edges. The head is mounted on a moulded handle. In some implementations of such a razor, a low friction strip, parallel to and above the razor edges, is integrated into the moulded shaver head to ease the friction of the shaving process.

Therefore, various means for lessening the skin irritation during shaving have been introduced. For example, U.S. Pat. Nos. 9,751,226; 9,707,688; 9,701,034; 9,694,503; 9,630,330; and 9,156,174 provide a lubricating cartridge attached through a retaining element to the razor head. However, several disadvantages were discovered in the use of the cartridges due to structural instability and variable retaining forces, as well as buckling. Manufacturing the cartridge has proven complex and expensive.

Compositions of various algae extracts are disclosed herein for skin care in view of the potential to provide enhanced hydration, anti-aging properties, UV protection, and prevent collagen and elastin fibers degradation amongst other benefits. Such algae extract compositions have multiple cosmetic activities for prevention of ageing, and for rejuvenation of mature skin. Algae useful in forming such compositions may be collected from the wild from specific pristine ecosystems often from the North Atlantic coast or from cultured sources preferably having highly controllable ambient conditions.

The object of the invention is to overcome the short comings of the plain unlubricated shaving head, and low friction strips of lubricated shaving heads. The shaving head is modified herein with at least one strip containing small lubricating material derived from algae extracts providing different benefits to the skin.

### SUMMARY OF THE INVENTION

There is disclosed herein a shaver comprising a head portion attached to a handle portion, wherein the head portion comprises a shaving blade unit having at least one blade with a cutting edge and having at least one lubricating strip attached to the head portion in parallel below the at least one blade, and, optionally, having a soap/foaming strip attached in parallel above the at least one blade.

The lubricating strip contains a flexible skin engaging material containing a composition of algae extracts comprising antioxidant and anti-inflammatory properties. The soap/foaming strip comprises skin moisturizing and restorative properties.

In another embodiment, the handle portion optionally is a hollow tube attached to the head portion filled with a balm composition disclosed herein and may be equipped with a balm dispensing wheel. Such balm may contain the algae extracts described herein.

A special aspect of the invention provides algae extracts comprising extracts, or mixtures thereof, from one or more of Laminaria ochroleuca, Alaria esculenta, Palmaria palmata and Ascophyllum nodosum.

A further aspect of the invention is directed to a shaver dispensing algae extracts as described herein, wherein algae extracts comprise an extract of Pelvetia canaliculata.

The shaver may contain an extract mixture containing an extract of Enteromorpha compressa.

Another aspect of the invention provides a shaver comprising an extract of Chondrus crispus.

Another aspect of the invention provides a shaver comprising an extract of Dunaliella salina.

Another aspect of the invention provides a shaver, wherein the extracts comprise at least one of Dunaliella salina, Chondrus crisps, Enteromorpha compressa and Pelvetia canaliculata.

### BRIEF DESCRIPTION OF THE DRAWINGS

FIG. 1 is an exemplary illustration of a razor head displaying an array of multiple blades equipped with a soap/foaming strip and lubricating strip; and

FIG. 2 is an exemplary perspective diagram of the components displaying a razor head and blades engaged with a handle comprising a balm rod capable of being capped with a lid, the position of the balm rod being adjustable by a rotating balm dispensing wheel.

### DETAILED DESCRIPTION OF THE INVENTION

The present invention generally relates to a system and method for ease of shaving comprising material for embedding and releasing compositions effective in treating and protecting skin during shaving or various other cosmetic applications or improvements. Algae extracts as topical ointments may be particularly useful for their antioxidant and anti-inflammatory properties.

Algae Extracton and Use

In the following embodiments there is disclosed a composition formed from a combination of extracts from several algae species. Of course one or more of such algae species may be employed in embodiments such compositions have multiple cosmetic activities for prevention of ageing and for rejuvenation of mature skin. Algae useful in forming such compositions may be collected from the wild from specific pristine ecosystems often from the North Atlantic coast or from cultured sources preferably having highly controllable ambient conditions.

Selected algal species useful in the formation of embodiment compositions include:
Pelvetia canaliculata often collected from the North Atlantic;
Laminaria ochroleuca often collected from the North Atlantic;
Enteromorpha compressa often collected from the North Atlantic;
Maria esculenta often collected from the North Atlantic;
Chondrus crispus often collected from the North Atlantic;
Dunaliella sauna cultured in photo-bioreactors;
Palmaria paimata often collected from the North Atlantic;
Ascophyliurn nodosum often collected from North Atlantic.

Algae Extracts:

1. In one embodiment, the extract mixture includes extracts from one or more of Laminaria ochroleuca, Alaria esculenta, Pa/maria palmata and Ascophyllum nodosum.

Another embodiment of the extract mixture contains all four algal species, or extracts from said algal species. Such an extract mixture may be used to prevent aging by modulating connective tissue (ECM) proteolysis in that it
(a) inhibits MMP1, 2, 3 & 9 and HLE over-expression preventing collagen and elastin fibers' degradation;
(b) stimulates collagen IV & VII synthesis;
(c) stimulates dermic trans-membrane proteoglycan synthesis;
(d) promotes skin rejuvenation by:

(i) down-regulating TGF-13, stimulating fibroblast proliferation, and

(ii) stimulating skin cell ATP synthesis.

Such an extract mixture may also be used to decrease chronic cell ageing (acts on keratinocytes, Langerhans cells; reduces skin inflammation and increase cell metabolism by reinforcing epidermis hydrolipidic barrier, decreasing LIVB induced inflammation mediators ILL, 1L6, TNFa, PGE2, LTB4, COX2, decreasing inflammation induced amplification of platelet activating factors on PAF receptors, protecting DNA against LiVB & UVA exposure). Anti-inflammatory activity of such extract mixtures prevents skin flush and ECM degradation by inhibiting dermis UVB induced monocyte transformation to macrophage at the same time promoting cutaneous collagen fibers and glycosaminoglycans (GAG) protection.

Such combination may also be used to promote healthy skin appearance and reduce pigmentation activity (polysaccharides rich in xylose and galactose limit melanogenesis i.e., limit tyrosinase activity and transport of melanosomes i.e. limit their protein anchoring complex of Rab27a & melanophilin, and control photo-induced pigmentation (limit production of the UVB exposure induced stem cell factor). Large molecular weight fucoidans aid skin miniaturization by forming a film on epidermis.

2. In embodiments, the extract mixture such as described in (1) further includes an extract of Peivetia canaikvlata:

This extract modulates ECM proteolysis by promoting ATP synthesis, proteoglycan synthesis, collagen I & collagen III synthesis, glycosaminoglycans (GAG) and hyaluronic acid synthesis. It also adds a strong anti-oxidant activity.

3. In embodiments, the mixture such as described in (1) includes Enteromorpha cotnpressa extract: This extract is rich in pentasaccharides, and reinforces synthesis of collagen I & III. The final solution has enhanced anti-oxidant and anti-microbial properties.

4. In another embodiment, the mixture such as described in (1) includes Chondrus crispus extract.

Carrageenan and mineral trace elements from this extract form a protective film over the skin by carrageenan binding to skin proteins (“second skin”) and resulting in skin softening and reduction of trans-epidermal water loss. Minerals (Mg, Ca) stimulate trans-glutaminase (Ca-dependent enzyme) activity leading to stronger extracellular matrix and enhanced skin firming.

5. In another embodiment, the mixture such as described in (1) includes an extract of Dunaliella sallna

This is enhancing anti-inflammatory and anti-oxidant activities, whilst the carotenoid content protects human skin from UV-induced erythema. It also adds antimicrobial, antiallergic and analgesic activities.

6. In another embodiment, the mixture such as described in (1) includes extracts of .Dunaliella saltha, Chondrus crispus, Enteromorpha compressa and Pelvetia canaliculata.

Overall, such combined mixtures exhibit the following properties:
Strong antioxidant (scavenging effects of superoxide and hydroxyl radicals etc., due to phenols and other compounds),
Membrane-protective (restoration of the lipid component of erythrocyte membranes and the removal of toxic stress),
stress-protective (extracts cause the normalization of stress accompanied by a decrease in osmotic resistance and antioxidant protection of erythrocytes),
photoprotective (due to phiorotannins and other compounds), immune-modulating effects (a significant increase in cytotoxic T cell numbers and phagocytic capacity in monocytes, and significant decrease in levels of the inflammatory cytokine interleukin 6; reduces skin inflammation (inhibiting the development of paw edema induced by k-carrageenan; suppressing TN1²-a and NO),
By its carotenoids protect humans from UV-induced erythema, anti-photo ageing (UVB-induced premature senescence of human skin diploid fibroblasts (HDFs), and provide whitening activity (inhibition ratio of tyrosinase activity of ethylacetate fraction; melanin production significantly inhibited in B16 melanoma cells);
anti-ageing incoldans (sulphated polysaccharides from brown algae) modulate connective tissue proteolysis and the effect of Transforming Growth Factor (TGF-beta) fibroblast proliferation and wound repopulation;
anti-microbial (antibacterial, anti-viral, antifungal activity against Malassezia species a major cause of dandruff and seborrheic dermatitis), and
anti-allergic.

Compositions of algae extracts are effective in a great variety of applications:

Shaving Products:

Help to achieve smooth and shiny skin, skin moisturization and nourishment, antioxidant and anti-inflammatory properties.

Cosmetic Primer:

Lightweight emulsion often used on eyes, lips or the face in general to act as a shield before the concealer or foundation is applied. It offers moisturization, sometimes UV protection and prepares the skin with conditioning elements in order to prevent damage from the rest of the makeup; also in some cases to even out skin tone.

Pore Minimising Products:

Lightweight and usually silicone based product, which fills in and evens out pores before foundation is applied.

Concealer:

Thick paste or liquid form of foundation. Used mainly to cover spots, severe discoloration, acne or scars, concealer would benefit from the extract mixture or its components with regards to moisturising, anti-aging benefit and also limit redness, discoloration, and fine lines.

Foundation:

Found in several different forms. Powder, stick, liquid spray or a newly developed formula called cushion (which basically uses a moist sponge that is soaked in the foundation, paste or liquid, which is preferred to cut down on product application. The extract mixture or its components would offer a moisturising, anti-aging benefit and also limit redness, discoloration, fine lines;
Eye-shadows:
   Paste and powder formulas;
Mascara
Brow products:
   Paste, liner, pencil, powder;
Lipstick/lip-gloss/lip treatment products, and
Makeup removing products:
   Both with leave-in and rinse-off formulae.

Benefits of the use of such compounds is to provide enhanced hydration, anti-aging properties, UV protection, and prevent collagen and elastin fibers degradation amongst other benefits. Skin care applications that could benefit from algae compounds comprise, without limitation:
Shaving products:
   Help to achieve smooth and shiny skin, skin moisturization and nourishment, antioxidant and anti-inflammatory properties.
Cosmetic primer:
   Lightweight emulsion often used on eyes, lips or the face in general to act as a shield before the concealer or foundation is applied. It offers moisturization, sometimes UV protection and prepares the skin with conditioning elements in order to prevent damage from the rest of the make up. Also in some cases even out skin tone.
Pore minimising products:
   Lightweight and usually silicone based product, which fills in and evens out pores before foundation is applied.
Concealer:
   Thick paste or liquid form of foundation. Used mainly to cover spots, severe discoloration, acne or scars, concealer would benefit from the extract mixture or its components with regards to moisturising, anti-aging benefit and also limit redness, discoloration, and fine lines.
Foundation:
   Algae extracts may be applied in several different forms. Powder, stick, liquid spray or a newly developed formula called cushion (which basically uses a moist sponge that is soaked in the foundation), paste or liquid, which is preferred to cut down on product application. The extract mixture or its components would offer a moisturising, anti-aging benefit and also limit redness, discoloration, fine lines.
Eye-shadows:
   Paste and powder formulas
Mascara
Brow products:
   Paste, liner, pencil, powder
Lipstick/lip-gloss/lip treatment products.
Makeup removing products:
   Both leave-in and rinse-off formula.

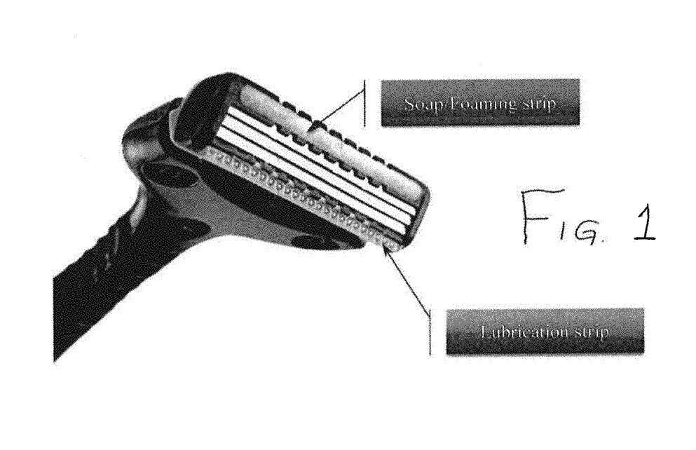

In a non-limiting embodiment, compounds formulated from algae may be applied to enhance the performance of shaving products. The present invention applies algae extracts to modify the lubricating strip of a razor head (FIG. 1).

Extracts that are deemed to be particularly applicable to serve as skin lubricating and anti-irritant ointment layer for the lubricating strip of a razor head. Extracts that present high temperature tolerance (temperatures around 200° C. for lubra strip (lubrication strip): process>2 extrusion stages or injection). The incorporated ingredients should have a beneficial effect on skin after thermal treatment.

Alaria spp. lipid extract was tested at 150° C. for lh and was found to retain a high degree its antioxidant/antiradical activity (2.456±0.175 ORAC test). Furthermore, it was proven resilient to degrading storage conditions (45 days at 45° C. and 5 days at UVB exposure) rendering it an ideal ingredient for commercial products that may be sustain such conditions at retail stores. Moreover, the extract scores high in a series of assays of cosmetic interest: >80% anti-elastase activity, >20% anti-collagenase activity, >20% hyaluronic acid synthesis induction, >20% collagen III synthesis induction, >20% skin matrix proteoglycans synthesis induction, >25% skin glucosamine glucans synthesis induction. In human volunteers the extract (in 1% cream preparations) resulted in >70% improvement of hydration levels and >90% skin firmness improvement. The extract is offered as lipid soluble liquid form with no preservatives used.

Extracts for Moisturization/Hydration, for Sensitive Skin

Chondrus crispus aqueous extracts offer extensive skin protection through hydration and conditioning, acting as second skin (natural silicone-like film forming). During clinical testing in human subjects, trans-epidermis water loss was reduced by about 40%, 2 h following application (2-3% cream preparation). The extract is a water soluble liquid with no preservatives added. Alaria spp. oily extracts also offer a combination of efficient skin hydration and improved skin firmness.

Extracts that Provide Lubricating Properties

For example, Chondrus crispus has a high carrageenan content and its extract has been characterized as “vegetable silicon” since it forms a film on the skin's surface which protects and reinforces the epidermis. In order to soothe irritation or prevent dryness of the skin, an alternative ingredient may be the Fucus spp. aqueous extract with anti-inflammatory, purifying and antiseptic properties.

Extracts that provide antibacterial properties (either on the material incorporated or on the skin).

Liquid extracts of Laminaria spp. Enteromorpha sp. Chondrus crispus, Dunaliella salina and other algae have shown in vitro antimicrobial properties. The extracts' antibacterial properties on the material may be applied after testing with standard methodology (shelf life etc.) in order to validate the desired effect.

Additionally, Asparagopsis sp. extract contains brominated and iodized compounds which delivered antimicrobial activity when used in 2% used cream preparations. This powdered extract is water soluble with no additives or preservatives.

Extracts for moisturezation/hydration for solid soaps (melt & pour), no constraints recording temperature here.

Alaria spp. oily extracts can be easily incorporated in the oil base of the soap substrate strip of the razor head, whereas Chondrus and Fucus aqueous extracts can be emulsified and added in the aqueous phase of the soap substrate.

Physical State of the Extracts:

All extracts are in the form of either aqueous or oily solutions except for the antibacterial extract which is in powder form.

### EXAMPLE A

### A First Shaving Device

A currently common shaving instrument is a disposable shaver as shown in FIG. 1. The disposable shaver typically comprises a moulded shaver head containing one or more parallel closely spaced razor edges. The head is mounted on a moulded handle. In some implementations, a low-friction strip, parallel to and above the razor blade edges, is integrated into the moulded shaver head.

In accordance with the teachings of this application, a dispenser strip may be positioned parallel to and below the razor blade edges. The dispenser strip may be supplied with the algae compound so as to distribute the compound onto the skin while in shaving motion. In one non-limiting embodiment, the disposable shaver may include a chamber to hold a supply of the algal compound. An internal conduit may be included to channel the compound from the chamber to the dispenser strip. Other dispensing means are also envisioned.

A non-limiting example of a first algal compound, suitable for shaving, having potentially strong anti-oxidant and anti-inflammatory properties is now described. The compound includes extracts of Alaria esculenta and Laminaria ochroleuca.

These extracts may be applied in different formulations for skin care, e.g. balms or lubrication parts of shaving apparatus. The Alaria extract may be applied at a range of 50-90% while that of Laminaria at a range of 5-25% The mixture may be used in liquid or dried form (i.e., spray-dried) as additives to balm formulations or lubrication formulations for shaving blades.

In case of the latter, the mixture of algae extracts is formulated together with known water-soluble (e.g. poly-ethylene oxide, polyacrylamide etc.) and non-water-soluble polymers (e.g. polystyrene) to be injection moulded or extruded to a final shape suitable to be mounted on a blade bearing razor head. The above-mentioned dried extracts used as additives, are partially rehydrated (between 30-60% per weight basis) prior to the moulding process and could withstand high temperature (over 150° C.), induced for a small period during the manufacturing process, without losing its desirable antioxidant efficacy. After the lubrication element is prepared, contact with water causes selective and proportional release of the additives enabling skin re-hydration and soothing during the shaving process.

### EXAMPLE 1

### Now Compound

A representative algae compound 1 may be manufactured as follows:
1. Weigh Alaria esculenta dry extract at 50-90% of final mixture
2. Weigh and mix the (1) with 5-25% of final weight dry extract of Laminaria
3. Mix well until lack of agglomerates or visible color differences (a mixing device could optionally be used (blender etc.)
4. Add water to the mixture up to 30-60% per weight if the final formulation method comprises a compression step involving heating (e.g. extrusion, injection moulding etc.) according to the desired lubrication formulation protocol selected or patented by the formulator
5. Add the water-soluble compound at room temperature or else according to the desired lubrication formulation protocol selected or patented by the formulator. If the water-soluble polymer is added in liquid form (preferred) then calculate the water addition of step (4) accordingly
6. Add the hydrophobic compound at room temperature or else according to the desired lubrication formulation protocol selected or patented by the formulator.
7. If the hydrophobic polymer is to be added in liquid form and if needed a suitable emulsifier should be added compatible to the specifications of the desired lubrication formulation protocol.
8. Mix well the preparation until no visible agglomeration preferably using medium to high shear if necessary (depends on other ingredients added by the formulator)
9. Apply compression according to the method provided by the desired lubrication formulation protocol selected by the formulator
10. Collect the ejected final product in shape and density required by the desired lubrication formulation protocol selected by the formulator

Testing of the final product for anti-oxidant and anti-inflammatory activity requires the product to be reasonably re-hydrated in a simulation of the shaving process to collect the aqueous extract and use a standard protocol (FRAPP etc.) by the formulator.

### EXAMPLE B

### A Second Shaving Device

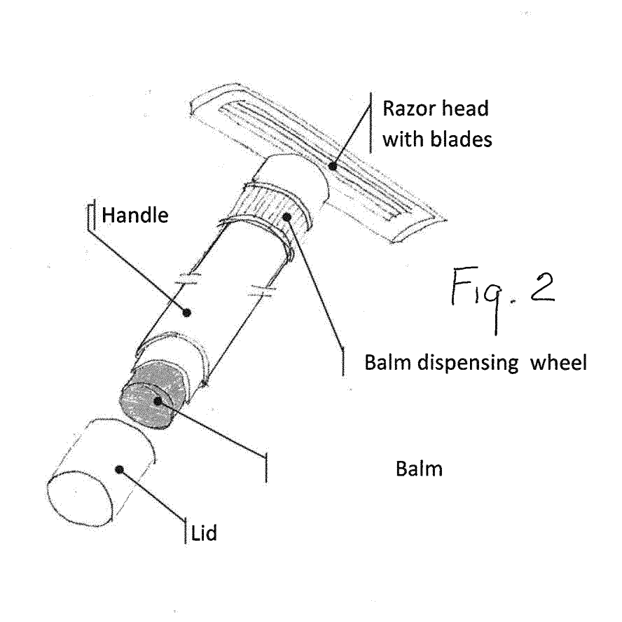

A second non-limiting embodiment of a shaver, as illustrated in FIG. 2, the device consists of two main reversibly linked portions. One end includes the shaver head and blades. The other holder end has a tube containing a balm which maybe infused with an algal compound, such as the first algal compound.

### EXAMPLE 2

### Algal Balm Compound I

The balm material I is created by melting basic ingredients such as beeswax, shea butter, coconut oil over boiling water and then when it is still warm (<40° C.) mix in first algal compound and essential oils to add fragrance. Once the mixture reaches desired consistency, it is poured into a tube (handle) and then fastened onto the shaving device.

### EXAMPLE 3

### Algal Balm Compound II

Another embodiment of the algal compound mixture 1 to Algal Balm II can be manufactured with the following method. Ingredients include Beeswax, Coconut Oil, Shea Butter, plant essential oils such as Peppermint Essential Oil, and first algal compound mixture 1 consisting of Alaria esculenta extract and Chondrus crispus extract.

20 g beeswax, 20 g shea butter and coconut oil are melted and mixed in a double boiler or small glass bowl over a small pot of boiling water, stirring constantly until melted. Bowl is removed from heat but kept over the still-hot water to keep the mixture melted. Add 1.5 ml of essential oils and 1 ml of the first algal compound mixture (1).

The amount produced is adequate for 12 to 14 balm dispensers depending on the dimensions of the combined razor-balm delivery device.

A tube handle-embedded balm (balm tube) may be accessed like a chap stick tube by twisting part of the balm dispensing wheel to reveal the balm. The balm tube can also be exchangeable and offered in refills to fit inside the razor handle.

### Other Embodiments

Other embodiments, such as skin massage and ultrasonic skin treatment devices can comprise a dispenser, within the head of the device, for applying the first algal compound to the skin.

In still further embodiments, applicators such as a washcloth, loofah, pumice stone, or disposable pad can be soaked with the first algal compound balm before it solidifies. Each time the customer uses the applicator a certain amount of balm will be delivered to the skin.

While the invention has been described with respect to preferred embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details, features and/or technical background.

What is claimed is:

1. A shaver comprising a head portion attached to a handle portion, wherein the head portion comprises a shaving blade unit having at least one blade with a cutting edge, having at least one lubricating strip attached to the head portion in parallel below the at least one blade, and, optionally, having a soap/foaming strip attached in parallel above the at least one blade; said lubricating strip comprising a flexible skin engaging material containing algae extracts comprising antioxidant and anti-inflammatory properties; and said soap/foaming strip comprising skin moisturizing and restorative properties; said handle portion optionally being a hollow tube attached to the head portion filled with an algae extract and balm composition and equipped with a balm dispensing wheel.

2. The shaver of claim 1, wherein algae extracts comprise extracts, or mixtures thereof, from one or more of Laminaria ochroleuca, Alaria esculenta, Palmaria palmata and Ascophyllum nodosum.

3. The shaver of claim 1, wherein algae extracts comprise an extract of Pelvetia canaliculata.

4. The shaver of claim 1, wherein extract mixture comprises an extract of Enteromorpha compressa.

5. The shaver of claim 1, wherein the extracts further comprise extract of Chondrus crispus.

6. The shaver of claim 1, wherein the extracts further comprise extract of Dunaliella salina.

7. The shaver of claim 1, wherein the extracts comprise at least one of Dunaliella salina, Chondrus crisps, Enteromorpha compressa and Pelvetia canaliculata.
